(19) United States
(12) Patent Application Publication
(10) Pub. No.: US 2012/0183619 A1
(43) Pub. Date: Jul. 19, 2012

(54) TRICLOSAN DERIVATIVES AND NANOPARTICLES COMPRISING SAME

(75) Inventors: Jean Paul LELLOUCHE, Ashdod (IL); Igor MAKAROVSKY, Holon (IL); Yonit BOGUSLAVSKY, Petach Tikva (IL); Ehud BANIN, Tel Aviv (IL); Jonatan LELLOUCHE, Ashdod (IL)

(73) Assignee: BAR ILAN UNIVERSITY, Ramat Gan (IL)

(21) Appl. No.: 13/285,516

(22) Filed: Oct. 31, 2011

Related U.S. Application Data
(60) Provisional application No. 61/457,154, filed on Jan. 18, 2011.

(51) Int. Cl. A01N 25/26; C07C 271/42; A01N 25/00; A01N 55/00; A01P 1/00; C07F 7/18; A01N 47/10; B82Y 99/00

(52) U.S. Cl. 424/490; 556/420; 560/157; 514/490; 424/400; 514/63; 977/773; 977/902

(57) ABSTRACT

The present invention is directed to triclosan derivatives and nanoparticles comprising said derivatives together with an organic or an inorganic carrier. The present invention is also directed to uses of the triclosan nanoparticles for preventing or inhibiting bacterial growth.

### FIELD OF THE INVENTION

The present invention is directed to triclosan derivatives and nanoparticles comprising said derivatives together with an organic or an inorganic carrier. The present invention is also directed to uses of the triclosan nanoparticles for preventing or inhibiting bacterial growth.

### BACKGROUND OF THE INVENTION

Triclosan (Irgasan®) is a well-known, commercial, Food and Drug Administration (FDA) approved, synthetic, non-ionic, broad-spectrum antimicrobial agent, possessing mostly antibacterial, but also some antifungal and antiviral properties. Triclosan is fairly insoluble in aqueous solution, unless the pH is alkaline, and is readily soluble in most organic solvents. It is chemically stable for up to 2 h at up to 200° C. The thermal stability makes triclosan suitable for incorporation into various reinforced plastic materials. Triclosan is used in many contemporary consumer and professional health-care products and is also incorporated into fabrics and plastics.

Nano-scale formulations of triclosan loaded into organic or inorganic matrices are designed to improve its delivery and bioavailability. These formulations also provide slow and controlled release of active compounds, increased water solubility, and improved stability of the active compound. For example, triclosan loaded into poly(D, L-lactideco-glycolide) (PLGA), poly(D, L-lactide) (PLA), and poly(vinyl alcohol) (PVAL) copolymers are disclosed in Kalyon B. D. et al. (J. Infection Control, 29, 124, 2011) and Pinon-Segundo E. et al. (Int. J. Pharm., 294, 217, 2005).

Mesoporous silica was previously utilized as a vehicle for the successful intracellular delivery of water insoluble or membrane-impermeable agents (Lu J. et al., 2007, Small, 3, 1341). Silica is an essential nutrient and plays an important role in many functions of living organisms, having a direct relationship to mineral absorption. Amorphous silica is nontoxic, biocompatible and biodegradable, freely dispersible throughout the body and ultimately excreted in the urine (Andersson J. et al., 2004, 16, 4160).

Another delivery system that may be applied as a biocide carrier is a polymer based acrylate. Acrylate polymers have gained much attention due to their biocompatibility and high water solubility. An acrylate polymer belongs to a group of polymers, which could be referred to generally as plastics. They are noted for their transparency and resistance to breakage and elasticity.

U.S. Pat. No. 6,464,961 discloses an oral care composition comprises acrylate polymer covalently bound to triclosan. The polymer-bactericide bond is hydrolytic labile ester bond, thus releasing the active compound in aqueous solution.

There is an unmet need for improved, especially stable, antimicrobial triclosan-based formulations.

### SUMMARY OF THE INVENTION

The present invention is directed to triclosan derivatives and nanoparticle compositions comprising same. The nanoparticles of the invention are based on triclosan derivatives bound to an inorganic or organic carrier. Preferably, the nanoparticles comprise a triclosan derivative covalently bound to silica or acrylate moieties.

The invention is also directed to methods for preventing bacteria growth using the triclosan nanoparticle compositions. The methods include contacting (e.g. coating, covering) the nanoparticle compositions with the surfaces of products, such as, health care products, fabrics, plastics, marine and medical equipment. The invention further provides methods for the treatment of diseases or disorders associated with bacteria growth using the triclosan nanoparticles.

The present invention is based in part on the unexpected discovery that both triclosan acrylate nanoparticles (“TA-NPs”) and triclosan silica nanoparticles (“T-SNPs”) present superior antibacterial activity, as compared to free triclosan. In addition, as opposed to free triclosan, the acrylate and silica nanoparticles of the invention are hydrophilic. The hydrophilic properties of the nanoparticles of the invention abrogate the need for solubilizing agents, which is of major advantage for any application. Advantageously, the nanoparticles of the invention are stable at aqueous solutions and do not confer their antibacterial activity unless activated.

Without wishing to be bound by any theory or mechanism, a covalent urethane bond between the carrier and the biocide renders great stability by preventing premature spontaneous release of the biocide in aqueous solution. Active triclosan may be released from the nanoparticles of the invention only upon exposure to bacterial enzymes, specifically esterases. This unique structure enables selective targeting of bacterial pathogen. As leaching of the toxic active agent from the nanoparticles composition of the invention is minimal the nanoparticles composition serves a non-toxic storage form of triclosan. Furthermore, since release of triclosan from the nanoparticles is controlled by enzymatic reactions, the nanoparticles can be designed for controlled release of triclosan.

According to a first aspect, the present invention provides a triclosan carbamate derivative. According to one embodiment, the triclosan carbamate derivative comprises a silane moiety bound to triclosan. According to another embodiment, the triclosan carbamate derivative comprises an isocyanate silane moiety. According to yet another embodiment, the triclosan carbamate derivative comprises 3-isocyanatopropyltriethoxysilane. According to yet another embodiment, the triclosan carbamate derivative consists of 5-chloro-2-(2,4-dichlorophenoxy)phenyl (3-(triethoxysilyl)propyl)carbamate (also termed hereinafter “triclosan-(3-(triethoxysilyl)propyl)carbamate”, “TTESPC” and “linker”).

According to yet another embodiment, the triclosan carbamate derivative is having a Fourier Transform Infra Red (FTIR) spectrum as set forth in FIG. 1.

According to yet another embodiment, the triclosan carbamate derivative is having a melting point of 83.5±1° C.

According to another aspect the present invention provides a composition comprising a plurality of nanoparticles, wherein each nanoparticle comprises a triclosan derivative and a carrier. According to one embodiment, the triclosan derivative is triclosan carbamate. According to another embodiment, the triclosan carbamate derivative comprises a silane moiety bound to triclosan. According to yet another embodiment, the triclosan carbamate derivative comprises 3-isocyanatopropyltriethoxysilane. According to yet another embodiment, the triclosan carbamate derivative consists of 5-chloro-2-(2,4-dichlorophenoxy)phenyl (3-(triethoxysilyl)propyl)carbamate.

According to yet another embodiment, the triclosan carbamate derivative is covalently bounds to the carrier. According to yet another embodiment, the carrier is an inorganic carrier. According to yet another embodiment, the inorganic carrier is a ceramic matrix carrier. According to yet another embodiment, the ceramic matrix is selected from the group consisting of: silica, glass, glaze, copper oxide, lead oxide, aluminum oxide, titanium oxide, zirconium oxide, aluminum nitride, titanium nitride, zirconium nitride, silicon carbide and a mixture thereof. Each possibility represents a separate embodiment of this invention.

According to yet another embodiment, the inorganic carrier is a solid silica matrix. According to yet another embodiment, the inorganic carrier is a mesoporous solid silica matrix.

The terms “silica shells”, “solid silica matrix” and “mesoporous solid silica matrix” are interchangeable and refer to hollow, typically ellipsoids, particles of highly porous silica, having a very high absorption capacity.

According to yet another embodiment, the inorganic carrier is positively charged. According to yet another embodiment, the inorganic carrier is aminated. According to yet another embodiment, the inorganic carrier comprises aminated silica.

According to yet another embodiment, the triclosan is released from the nanoparticles in a modified release manner. According to yet another embodiment, the triclosan is released from the triclosan carbamate derivative by enzymatic cleavage. According to yet another embodiment, the triclosan is released in a slow release manner. According to another embodiment, the bond between the triclosan carbamate derivative and the inorganic carrier is not spontaneously hydrolysable in aqueous solution.

According to yet another embodiment, the triclosan carbamate derivative and the inorganic carrier remain bound in pH within the range of 6 to 10.

According to yet another embodiment, the amount of triclosan within each nanoparticle is within the range of 0.25 to 1.0 wt % relative to the total weight of the nanoparticle.

According to yet another embodiment, the triclosan derivative is covalently bound to the inorganic carrier via a hydrolytically labile urethane bond.

According to an alternative embodiment, the composition comprises a plurality of nanoparticles, wherein each nanoparticle comprises a triclosan acrylate polymer. According to another embodiment, the polymer comprises triclosan acrylate monomers. According to yet another embodiment, the triclosan binds the acrylate moiety via an ester bond.

According to yet another embodiment, the triclosan is not released from the nanoparticles spontaneously, e.g. via hydrolysis upon contact with aqueous solutions. According to yet another embodiment, triclosan is released from the nanoparticles by enzymatic cleavage.

It is to be understood that by the term ‘released from the nanoparticles’ with reference to triclosan, it is intended to state that substantial amounts are of triclosan are released from the nanoparticles where ‘substantial amounts’ refer to triclosan amounts that exert a significant toxic reaction, which is at least above detection levels.

According to yet another embodiment, the plurality of nanoparticles exhibit a particle size distribution within the range of 30 nm to 200 nm in diameter, within the range of 80 nm to 200 nm, within the range of 30 nm to 150 nm or within the range of 80 nm to 150 nm. Each possibility represents a separate embodiment of this invention.

According to yet another embodiment, the nanoparticle composition is for inhibiting, attenuating or preventing microbial growth.

According to yet another aspect, the present invention provides a method for inhibiting microbial growth comprising contacting a surface with a composition comprising a plurality of triclosan nanoparticles, each nanoparticle comprises triclosan derivative and a carrier.

According to one embodiment, the surface is a surface of products selected from the group consisting of: heath care products, fabrics, plastics, marine equipment and medical equipment. Each possibility represents a separate embodiment of this invention.

According to another embodiment, inhibiting the growth is attenuating the growth. According to yet another embodiment, inhibiting the growth is preventing the growth. According to yet another embodiment, inhibiting the growth is causing complete or partial cell lyses. According to yet another aspect the present invention provides a method for treating a disease or disorder in a mammalian subject in need thereof, comprising administering a composition comprising a plurality of nanoparticles, wherein each nanoparticle comprises triclosan derivative and a pharmaceutically acceptable carrier and wherein the disease or disorder are associated with bacterial growth.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A is a Fourier Transform Infrared (FTIR) spectrum of triclosan-(3-(triethoxysilyl)propyl) carbamate (TTESPC).

FIG. 1B is an FTIR spectrum of the triclosan silica nanoparticles (T-SNPs).

FIG. 2 is a scheme demonstrating the chemical structure of triclosan (Irgasan®).

FIG. 3 is a scheme demonstrating the synthetic pathway for the fabrication of triclosan silica nanoparticles.

FIG. 4 presents the UV spectra of (a) TTESPC, (b) Triclosan, (c) T-SNPs, and (d) bare SNPs, all in ethanol.

FIG. 5 presents the size distribution of T-SNPs.

FIG. 6 shows the pH dependency of the potential (A, dashed line) and the average size (B, solid line) of T-SNPs.

FIG. 7A shows the effect of the initial TTESPC concentration on the average T-SNPs diameter, as measured by Dynamic Light Scaterring (DLS), at 25° C. (a, dashed line) and at 60° C. (b, solid line).

FIG. 7B shows the effect of the initial TTESPC concentration on triclosan content within T-SNPs, as calculated from elemental-analysis data, at 25° C. (a, dashed line) and at 60° C. (b, solid line).

FIG. 8 is a TGA thermogram of T-SNPs (curve a) and DSC thermograms of triclosan (curve b), TTESPC (curve c) and T-SNPs (curve d).

FIG. 9A is an HR-SEM micrograph of T-SNPs (scale: 100 nm).

FIG. 9B is a TEM micrograph of T-SNPs (scale: 500 nm).

FIG. 10 shows the antimicrobial activity of T-SNPs on E. coli (A) and on S. aureus (B).

FIG. 11 presents the minimal inhibitory concentration (MIC) of triclosan on E. coli. (A) and on S. aureus (B).

FIG. 12 shows TEM micrographs of untreated E. coli (A) and S. aureus (B), and of T-SNPs treated E. coli (C) and S. aureus (D). E-F) Magnified images of the adjacent micrographs (C and D).

FIG. 13 is a scheme of a polymer of triclosan acrylate nanoparticles (TA-NPs).

FIG. 14A is a HR-SEM micrograph of TA-NPs (scale: 100 nm).

FIG. 14B is a TEM micrograph of TA-NPs (scale: 500 nm).

FIG. 15 is a scheme demonstrating the chemical structure of aminated hybrid silica nanoparticles containing triclosan (NH₂-T-SNPs).

FIG. 16 is a TEM micrograph (scale bar: 200 nm) of NH₂-T-SNPs.

FIG. 17 shows the antimicrobial activity of NH₂-T-SNPs on E. Coli (A) and on S. aureus (B).

FIG. 18 shows the antimicrobial activity of TA-NPs on E. Coli (A) and on S. aureus (B).

### DETAILED DESCRIPTION OF THE INVENTION

The present invention is directed to triclosan derivatives and nanoparticles comprising same, wherein the triclosan derivative is covalently linked to an inorganic or organic carrier, such that triclosan release is facilitated primarily upon enzymatic reaction.

### Triclosan Derivatives

The present invention provides a triclosan carbamate derivative. According to one embodiment, the triclosan carbamate derivative comprises a silane moiety bound to triclosan. According to another embodiment, the triclosan carbamate derivative comprises an isocyanate silane moiety. According to yet another embodiment, the triclosan carbamate derivative comprises a 3-isocyanatopropyltriethoxysilane moiety. According to yet another embodiment, the triclosan derivative is 5-chloro-2-(2,4-dichlorophenoxy)phenyl (3-(triethoxysilyl)propyl)carbamate, also termed hereinafter “triclosan 3-isocyanatopropyltriethoxysilane”, “TTESCP” or “linker”.

The term “derivatives of triclosan” as used herein include those compounds in which one or both of the phenyl groups is/are substituted by one or more substituent groups in addition to the chloro substituents already present on the phenyl rings. Examples of suitable substituents are halogens, including, but not limited to, F, Br and Cl, alkyl groups containing 1 to 4 carbon atoms, haloalkyl groups containing 1 to 4 carbon atoms, alkoxy group containing 1 to 4 carbon atoms, cyano, allyl, amino and acetyl groups. Preferred substituents are halogen atoms, such as, F, Br and Cl. It will be understood that if triclosan is substituted by more than one substituent, then the substituents may be the same or different.

The term “triclosan carbamate derivative” as used herein is intended to encompass any carbamate derivative of triclosan, including derivatives of triclosan as defined hereinabove and the cationic salts of triclosan carbamate derivatives.

In some embodiment, the triclosan carbamate derivative is in the form of a cationic salt. In other embodiments, the cationic salt is selected from the group consisting of: sodium salt, potassium salt, calcium salt and magnesium salt.

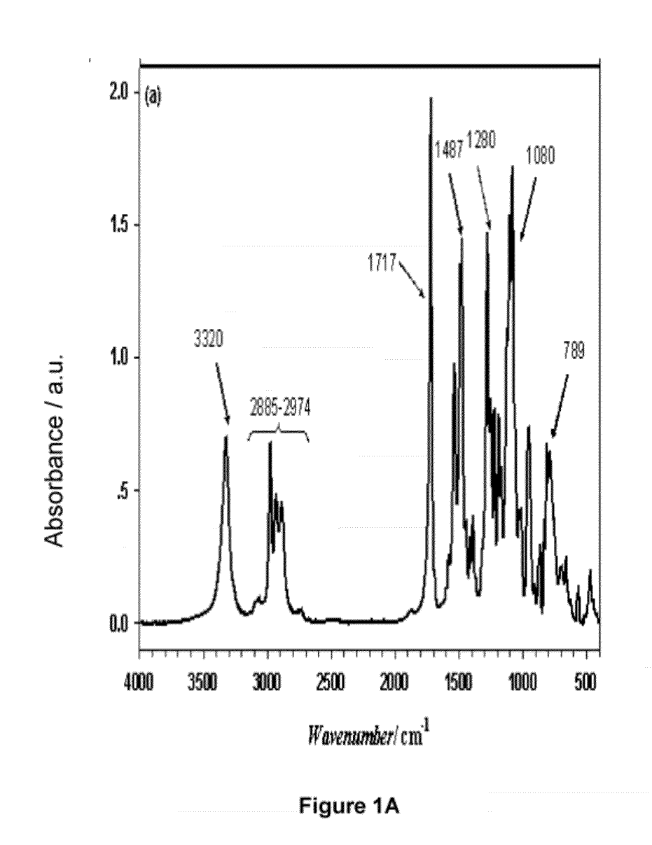

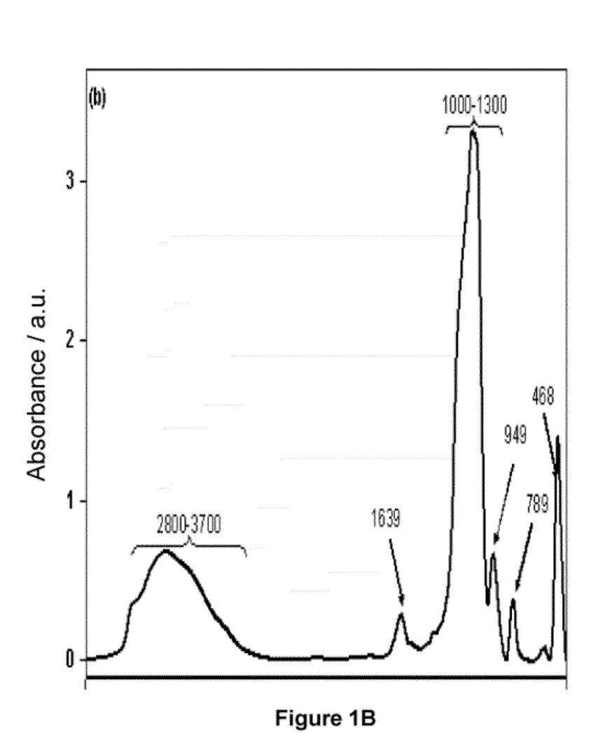

In further embodiments, the triclosan-carbamate derivative of the invention is characterized by one or more of the following properties:
(a) a Fourier Transform Infra Red (FTIR) spectrum as set forth in FIG. 1;
(b) a melting point of 83.5° C.±1° C.;
(c) having an ¹H NMR comprising bands in one or more of the following chemical shifts (300 MHz, CDCl₃, 25° C., TMS, δ): 7.43 (d, J=2.5 Hz, 1H, ClCCHCl), 7.26 (d, J=2.5 Hz, 1H, ClCCHC(O)C(O)), 7.15 (dd, J=8.8, 2.5 Hz, 1H, ClCCHCHC(O)Cl), 7.13 (d, J=2.5 Hz, 1H, ClCCHCHC(O)C(O)), 6.87 (d, J=8.8 Hz, 1H, ClCCHCHC(O)C(O)), 6.83 (d, J=8.8 Hz, 1H, ClCCHCHC(O)Cl), 5.36 (bt, 1H, NH), 3.81 (q, J=7.0 Hz, 6H, CH₃CH₂O), 3.20 (m, 2H, NHCH₂), 1.63 (m, 2H, NHCH2C H₂), 1.21 (t, J=7.0 Hz, 9H, CH₃CH₂O), 0.62 (m, 2H, SiCH₂);
(d) having a ¹³C NMR comprising bands in one or more of the following chemical shifts (75.5 MHz, CDCl₃, 25° C., TMS, 6): 153.2 (1C, NHC═O(O)), 151.4 (1C, CHC(O)C(O)), 147.0 (1C, ClC(O)CH), 142.3 (1C, NHCO₂C), 130.2 (1C, ClCCHCl), 129.3 (1C, ClCCHC(O)), 129.1 (1C, ClCCHCHC(O)Cl), 128.1 (1C, ClCCHCHC(O)Cl), 126.4 (1C, ClCCHCHC(O)C(O)), 125.6 (1C, ClCC(O)CH), 124.8 (1C, ClCCHC(O)), 120.4 (1C, ClCCHCHC(O)Cl), 120.2 (1C, ClCCHCHC(O)C(O)), 58.5 (3C, CH₃CH₂O), 43.6 (1C, NHCH₂), 22.8 (1C, NHCH₂CH₂), 18.2 (3C, CH₃CH₂O), 7.5 (1C, SiCH₂);
(e) having the IR spectrum (KBr) comprising the following wavenumbers: ν=3320 (m; ν as (NH)), 2974 (m; ν as (CH₂)), 2927 (m; ν as (CH₂)), 2885 (m; ν as (CH₂)), 1717 (vs; ν (C═O)), 1534 (s), 1487 (s; ν as (aromatic C═C)), 1474 (vs), 1389 (w), 1280 (vs; ν as (SiOC)), 1250 (m), 1219 (m), 1187 (m), 1080 (vs; vs (phenolic CO)), 956 (m; ν as (aromatic CH)), 789 cm−1 (m; ν as (Cl)); and
(f) having a UV-vis spectrum of λ max (ε)=276 (2822), 230 (17 407), 209 nm (32 160); and
(g) having the following mass spectra (MS) characteristics: CIMS (m/z (%)) 536.08 (M+, 5.48), 489.98 (C₂₀H₂₃Cl₃NO₅ Si., 100.00); HRMS (ESI, m/z): [M+H]+calculated for C₂₂H₂₈Cl₃NO₆ Si, 535.905; found, 536.085.

### Nanoparticles of Triclosan Derivatives

The present invention provides a composition comprising a plurality of nanoparticles, wherein each nanoparticle comprises a triclosan derivative covalently bound to a carrier.

The nanoparticles of the invention are stable at aqueous solutions and do not confer their antibacterial activity unless activated. In fact, the nanoparticles of the invention are pathogen-activated. Upon being consumed or otherwise internalized by a pathogen, the toxic triclosan is released from the inert nanoparticle structure. Triclosan release is afforded by enzymatic reaction(s) exerted by the pathogen's own enzymes. Once released, triclosan applies its toxic activity on said pathogen. Thus, the nanoparticles of the invention provide an inert (non toxic) storage platform for a toxic agent, that is stable at wide pH and temperature ranges, and which becomes active (toxic) only when disinfection activity is required, namely, only upon contact with microbes. This mechanism of action is an important advantage of the nanoparticles of the invention.

According to one embodiment, the carrier is an inorganic carrier such as an inorganic ceramic matrix carrier. According to another embodiment, the ceramic matrix is selected from the group consisting of: silica, glass, glaze, copper oxide, lead oxide, aluminum oxide, titanium oxide, zirconium oxide, aluminum nitride, titanium nitride, zirconium nitride, silicon carbide and a mixture thereof. Each possibility represents a separate embodiment of this invention.

According to yet another embodiment, the inorganic carrier is a solid silica matrix. According to yet another embodiment, the inorganic carrier is a mesoporous solid silica matrix.

As used herein the term “mesoporous” refers to silica matrices that possess adjustable pore sizes within the range of 1.5 to 10.0 nm.

According to yet another embodiment, the nanoparticles are positively charged. According to yet another embodiment, the nanoparticles further comprise positively charged groups. According to yet another embodiment, the positively charged groups are linked onto the surface of the nanoparticles. According to yet another embodiment, the nanoparticles comprise triclosan derivatives, an inorganic carrier, and a positively charged moiety selected from the group consisting of: polyethyleneimine (PEIs), polyglutamic species, positively charged polysaccharides of various molecular weights, such as, chitosan, and silicate derivatives thereof. According to yet another embodiment, the nanoparticles comprise triclosan derivatives and polyaminated silica shells.

According to yet another embodiment, the triclosan derivative of the invention and the carrier remain bound within a broad pH range. According to yet another embodiment, the triclosan derivative of the invention and the carrier remain bound in a pH within the range of 6 to 10.

According to an alternative embodiment, the nanoparticle composition of the invention comprises a plurality of nanoparticles wherein each nanoparticle comprises triclosan acrylate polymers. According to yet another embodiment, the polymer comprises triclosan acrylate monomers.

As used herein, the term “polymer” refers to a plurality of repeating structural units (backbone units) covalently connected to one another. This term encompasses organic and inorganic polymers and further encompasses one or more of a homopolymer, a copolymer or a mixture thereof (a blend). The polymers may be of any molecular weight. The term “homopolymer” as used herein refers to a polymer that is made up of one type of monomeric units and hence is composed of homogenic backbone units. The term “copolymer” as used herein refers to a polymer that is made up of more than one type of monomeric units and hence is composed of heterogenic backbone units. The heterogenic backbone units can differ from one another by the pendant groups thereof. In one embodiment, the polymer comprises of backbone comprised of units formed by polymerizing the corresponding monomeric units whereby the antimicrobial agent is attached to at least a portion of these backbone units. In another embodiment the polymer can be a synthetic polymer or a naturally occurring polymer. In yet another embodiment, the polymer is a synthetic polymer. In yet another embodiment, the polymeric backbone is selected from the group consisting of polyvinyls, polyamides, polyurethanes, polyimines, polysaccharides, polypeptides, polycarboxylates, and mixtures thereof. Each possibility represents a separate embodiment of this invention. Exemplary polymers include, but are not limited to a water soluble polyamino acid, a polyethyleneglycol (PEG), a polyglutamic acid (PGA), a polylactic acid (PLA), a polylactic-co-glycolic acid (PLGA), a poly(D,L-lactide-co glycolide) (PLA/PLGA), a poly(hydroxyalkyl-methacrylamide), a polyglycerol, a polyamidoamine (PAMAM), and a polyethylenimine (PEI). Each possibility represents a separate embodiment of this invention.

In yet another embodiment, the polymeric backbone is derived from polyacrylate or a copolymer thereof. In yet another embodiment, the polymeric backbone comprises acrylate backbone units having attached thereto either acrylate groups or such acrylate groups that have been modified by attaching thereto the biocide described herein.

In yet another embodiment, the polymeric nanoparticles described herein are composed of a polymeric backbone, formed from a plurality of backbone units that are covalently linked to one another, wherein at least a portion of this plurality of backbone units has an antimicrobial agent, as described herein, and attached thereto. The polymeric backbone can further include non-functionalized backbone units, as discussed hereinbelow, to which no antimicrobial agent is attached.

According yet another embodiment, the amount of triclosan within each nanoparticle is within the range of 0.25 to 1.0 wt % relative to the total weight of the nanoparticle.

As used herein, the term “weight percent (wt %)” refers to the concentration of the substance as the weight of that substance divided by the weight of the nanoparticle and multiplied by 100. The wt % loading may be measured by methods well known by those skilled in the art, some of which are described herein below under the Examples section that follows.

As used herein the term “nanospheres” refers to nanoparticles having a spherical shape which can be characterized by diameter or radius.

According to yet another embodiment, the nanoparticles exhibit a particle size distribution within the range of 1 nm to 1000 nm, from 1 nm to 800 nm, from 1 nm to 600 nm, from 1 nm to 400 nm, from 1 nm to 200 nm.

According to yet another embodiment, the nanoparticles exhibit a particle size distribution within the range of 30 nm to 200 nm in diameter.

According to yet another embodiment, the nanoparticles exhibit a particle size distribution within the range of 80 nm to 200 nm in diameter.

According to yet another embodiment, the nanoparticles exhibit a particle size distribution within the range of 30 nm to 150 nm in diameter.

According to yet another embodiment, the nanoparticles exhibit a particle size distribution within the range of 80 nm to 150 nm in diameter.

The term “particle size” as used herein typically refers to particle size evaluated for a spherical object and thus is defined by its diameter. However, the shape of typical particles may also be irregular and non-spherical. Thus, the quantitative definition of particle size as used herein is adjusted such that it also applies to non-spherical particles. It is important to note that the common definitions for particle size are based on replacing a given particle with an imaginary sphere having one of the properties identical with the particle. These properties include: volume, weight, area or a drag coefficient (a dimensionless number characterizing the overall drag of an object). For particles with sizes below a micrometer the definition is more complex since for small particle thickness of interface layer becomes comparable with the particle size. As a result, position of the particle surface becomes uncertain. The particle size for an ensemble (collection) of particles presents another problem. In real systems the particles are usually ensembles having different sizes and there is often a need of a certain average particle size for the ensemble of particles. The various average sizes include median size, geometric mean size and average size.

Several methods for measuring particle size are known in the art. The methods are based on light, ultrasound, electric field, gravity, or centrifugation.

The terms “particle size distribution” (PSD), is used herein to describe a list of values or a mathematical function that define the average particle size obtained for a sample of particles, sorted according to size (e.g. weight, volume, diameter) in a powder, granular material, or particles dispersed in fluid. It is important to note that PSD is usually defined by the method by which it is determined and only applies on a representative sample. PSD may be expressed as a “range” analysis, in which the amount in each size range is listed in order or in “cumulative” form, in which the total of all sizes “retained” or “passed” by a single parameter is given for a range of sizes. Range analysis is usually used when a particular ideal mid-range particle size is required and cumulative analysis is typically used where the amount of “under-size” or “over-size” must be controlled.

Measurement techniques include sieve analysis, air elutriation analysis, photoanalysis, electroresistance counting methods, sedimentation techniques, laser diffraction methods, acoustic spectroscopy or ultrasound attenuation spectroscopy and optical counting methods among others. Each possibility represents a separate embodiment of this invention.

In sieve analysis the powder is separated on sieves of different sizes and the PSD is defined in terms of discrete size ranges based on the sizes of the sieves that are used. The PSD is usually determined over a list of size ranges that covers nearly all the sizes present in the sample. Some methods of determination allow much narrower size ranges to be defined than can be obtained by use of sieves, and are applicable to particle sizes outside the range available in sieves. This method is simple, cost effective, and easily interpreted. However, many PSDs are concerned with particles too small for separation by sieving to be practical since the very fine sieves are fragile. In addition, the amount of energy used to sieve the sample is arbitrarily determined where over-energetic sieving causes attrition of the particles and thus changes the PSD, while insufficient energy fails to break down loose agglomerates.

Methods that are often dominant in industrial PSD determination are the laser diffraction methods, which depend on analysis of the “halo” of diffracted light produced when a laser beam passes through a dispersion of particles in air or in a liquid. The angle of diffraction increases as particle size decreases, so that this method is particularly good for measuring sizes between 0.1 and 3,000 μm. Advanced sophisticated data processing and automation allows this to be a suitable industrial method.

The nanoparticles of the present invention can be water-soluble or water-insoluble. In one embodiment, the nanoparticles are water soluble. In another embodiment, the nanoparticles can be charged nanoparticles or non-charged nanoparticles. Charged nanoparticles can be cationic nanoparticles, having positively charged groups and a positive net charge at a physiological pH; or anionic polymers, having negatively charged groups and a negative net charge at a physiological pH. Non-charged nanoparticles can have positively charged and negatively charged group with a neutral net charge at physiological pH, or can be non-charged.

Pharmaceutical Compositions

According to another aspect the present invention provides a pharmaceutical composition comprising, as an active ingredient, any of the nanoparticles described herein and a pharmaceutically acceptable carrier. Accordingly, in any of the methods and uses described herein, any of the nanoparticles described herein can be provided to an individual either per se, or as part of a pharmaceutical composition where it is mixed with a pharmaceutically acceptable carrier.

As used herein a “pharmaceutical composition” refers to a preparation of one or more of the nanoparticles described herein (as active ingredient), or physiologically acceptable salts thereof, with other chemical components including but not limited to physiologically suitable carriers, excipients, lubricants, buffering agents, antibacterial agents, bulking agents (e.g. mannitol), antioxidants (e.g., ascorbic acid or sodium bisulfate), anti-inflammatory agents, anti-viral agents, anti-histamines and the like. The purpose of a pharmaceutical composition is to facilitate administration of a compound to a subject, preferably, topical administration on an external surface (e.g. skin and teeth).

The term “active ingredient” as used herein refers to a compound, which is accountable for a biological effect.

The terms “physiologically acceptable carrier” and “pharmaceutically acceptable carrier” are interchangeably used to describe a carrier or a diluent that does not cause significant irritation to an organism and does not abrogate the biological activity and properties of the administered compound.

Herein the term “excipient” refers to an inert (biologically inactive) substance added to a pharmaceutical composition to further facilitate administration of a drug. Examples, without limitation, of excipients include various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present invention thus may be formulated in conventional manner using one or more pharmaceutically acceptable carriers comprising excipients and auxiliaries, which facilitate processing of the compounds into preparations, which can be used pharmaceutically. Proper formulation is dependent upon the route of administration chosen. The dosage may vary depending upon the dosage form employed and the route of administration utilized. The exact formulation, route of administration and dosage can be chosen by the individual physician in view of the patient's condition (see e.g., Fingl et al., 1975, in “The Pharmacological Basis of Therapeutics”, Ch. 1 p. 1).

The pharmaceutical composition may be formulated for administration in either one or more of routes depending on whether local or systemic treatment or administration is of choice, and on the area to be treated. Formulations for topical administration may include but are not limited to lotions, ointments, gels, creams, suppositories, drops, liquids, sprays and powders. Conventional pharmaceutical carriers, aqueous, powder or oily bases, thickeners and the like may be necessary or desirable.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice for example, may be of labeling approved by the U.S. Food and Drug Administration for prescription drugs or of an approved product insert. In any of the methods, uses and compositions described herein, the nanoparticles described herein can be utilized in combination with additional therapeutically active agents. Such additional agents include, as non-limiting examples, chemotherapeutic agents, anti-angiogenesis agents, hormones, growth factors, antibiotics, anti-microbial agents, anti-depressants, immunostimulants, and any other agent that may enhance the therapeutic effect of the nanoparticles and/or the well being of the treated subject.

### Methods for Inhibiting Microbial Growth

The present invention further provides method for inhibiting, attenuating and preventing microbial growth using a composition comprising a plurality of nanoparticles, wherein each nanoparticle comprises triclosan derivative and an inorganic or organic carrier.

The methods of the invention are targeted towards microorganisms, also termed herein “microbs”. The microorganism may be a microorganism selected from the group consisting of: algae, fungi, bacteria, parasites, protozoans, archaea, protests, amoeba and mold. Each possibility represents a separate embodiment of the present invention. Microorganisms according to the principals of the present invention include but are not limited to Staphylococcus epidermidis, Escherichia coli, Cellulophaga lytica, Navicula incerta, Halomonas pacifica, Pseudoalteromonas atlantica, Cobetia marina, Candida albicans, Clostridium difficile, Listeria monocytogenes, Staphylococcus aureus, Streptococcus faecalis, Bacillus subtilis, Salmonella chloraesius, Salmonella typhosa, Mycobacterium tuberculosis, Pseudomonas aeruginosa, Aerobacter aerogenes, Saccharomyces cerevisiae, Aspergillus niger, Aspergillus flares, Aspergillus terreus, Aspergillus verrucaria, Aureobasidium pullulans, Chaetomium globosum, Penicillum funiculosum, Trichophyton interdigital, Pullularia pullulans, Trichoderm sp. madison P-42, Cephaldascus fragans; Chrysophyta, Oscillatoria borneti, Anabaena cylindrical, Selenastrum gracile, Pleurococcus sp., Gonium sp., Volvox sp., Klebsiella pneumoniae, Pseudomonas fluorescens, Proteus mirabilis, Enterobacteriaceae, Acinetobacter spp., Pseudomonas spp., Candida spp., Candida tropicalis, Streptococcus salivarius, Rothia dentocariosa, Micrococcus luteus, Sarcina lutea, Salmonella typhimurium, Serratia marcescens, Candida utilis, Hansenula anomala, Kluyveromyces marxianus, Listeria monocytogenes, Serratia liquefasciens, Micrococcus lysodeikticus, Alicyclobacillus acidoterrestris, MRSA, Bacillus megaterium, Desulfovibrio sulfuricans, Streptococcus mutans, Cobetia marina, Enterobacter aerogenes, Enterobacter cloacae, Proteus vulgaris, Proteus mirabilis, Lactobacillus plantarum, Halomonas pacifica, and Ulva linza.

The invention further provides methods of reducing, inhibiting or preventing microbial growth or biofilm formation on a surface.

These methods include applying onto, coating, covering or otherwise contacting, the surface with the antimicrobial nanoparticles compositions of the invention. The surface may be marine surface, including, but not limited to, boat or ship hulls, anchors, docks, jetties, sewage pipes and drains, fountains, water-holding containers or tanks, and any surface in contact with a freshwater or saltwater environment. The surface may be a medical surface, including but not limited to, implants, medical devices, examination tables, instrument surfaces, knobs, handles, rails, poles, countertops, sinks, and faucets. Implants and medical devices include, but are not limited to, prosthetic heart valves, urinary catheters, venous catheters, endotracheal tubes, and orthopedic implants. The surface may also be a household surface including, but not limited to, countertops, sink surfaces, cupboard surfaces, shelf surfaces, knobs, handles, rails, poles, countertops, sinks, and faucets. The nanoparticles composition of the invention may be in the form of paint, such as a marine paint

The term “biofilm” typically refers to layers of proteins, DNA, and polysaccharides produced by microorganisms, and cells of the microorganisms themselves.

Determining the effect of a biocide, such as the triclosan nanoparticles of the invention, on microorganism may include calculating the minimal inhibitory concentration (MIC) on said biocide. MIC is the lowest concentration of an antimicrobial that inhibits the biocide growth of microorganisms following overnight incubation. Antimicrobial activity of antimicrobial compositions may be determined by any method known in the art, including as described in Examples 3FIG. 11.

The present invention also provides a method for treating a disease or disorder, comprising administering an effective amount of the composition comprising the nanoparticles of the invention to a subject in need thereof.

According to one embodiment, the disease or disorder are associated with bacteria growth. In another embodiment, the disease is Malaria. Malaria is caused by several species of the protozoan such as, Plasmodium, P. vivax and P. falciparum. They all have complex life cycles involving both the Anopheles mosquito and the erythrocyte of the human host.

The term “subject” as used herein refers to an animal, preferably a mammal, most preferably a human, who has been the object of treatment, observation or experiment.

### Processes for Preparing the Nanoparticles

The nanoparticles of the invention may be prepared by a sol-gel-based process. Partly hydrolysed oxides of suitable metals (including transition metals, silicon, etc.) prepared in the presence of an active material by hydrolysis of the gel precursor followed by condensation (alternatively referred to as polycondensation). The gel precursor may be a metal oxide gel precursor including silicon oxide gel precursor, transition metal oxide precursor, etc. The identity of the gel precursor chosen that is, whether a silicon oxide gel precursor or a particular metal oxide gel precursor chosen for use in a process of the invention, will depend on the intended use of the ceramic particles and, in particular, the suitability of the final product resulting from the condensation of the gel precursor for the intended use of the ceramic particles. The gel precursor is typically a silica-based gel precursor, an alumina-based gel precursor, a titanium dioxide-based gel precursor, an iron oxide based gel precursor, a zirconium dioxide-based gel precursor or any combination thereof. A functionalised, derivatised or partially hydrolysed gel precursor may be used. For silica there is a long list of potential silicon precursors which for convenience can be divided into 4 categories, the silicates (silicon acetate, silicic acid or salts thereof) the silsequioxanes and poly-silsequioxanes, the silicon alkoxides (from silicon methoxide (C1) to silicon octadecyloxide (C18)), and functionalised alkoxides for ORMOCER® production (such as ethyltrimethoxysilane, aminopropyltriethoxysilane, vinyltrimethoxysilane, diethyldiethoxysilane, diphenyldiethoxysilane, etc). Further specific examples of silica-based gel precursors include tetramethoxysilane (TMOS), tetraethoxysilane (TEOS), tetrabutoxysilane (TBOS), tetrapropoxysilane (TPOS), polydiethoxysilane, methyltrimethoxysilane, methyltriethoxysilane, ethyltriethoxysilane, octylpolysilsesquioxane and hexylpolysilsesquioxane.

The silica gel precursor or the metal oxide gel precursor may include from one to four alkoxide groups each having from 1 or more oxygen atoms, and from 1 to 18 carbon atoms, more typically from 1 to 5 carbon atoms. Alkoxide groups may be replaced by one or more suitable modifying groups or functionalised or derivatised by one or more suitable derivatizing groups.

Typically, the silica gel precursor is a silicon alkoxide or a silicon alkyl alkoxide. Particular examples of suitable silicon alkoxide precursors include such as methoxide, ethoxide, iso-propoxide, butoxide and pentyl oxide. Particular examples of suitable silicon or metal alkyl (or phenyl) alkoxide precursors include methyl trimethoxysilane, di-methyldimethoxysilane, ethyltriethoxysilane, diethyldiethoxysilane, triethyl-methoxysilane, phenyltriethoxysilane, diphenyldiethoxysilane, vinyltriethoxysilane, etc. Alternatively, the silica gel precursor may be a silicon carboxylate. For example, an acetate, tartrate, oxalate, lactate, propylate, formate, or citrate forms. Examples of other functional groups attached to silica gel precursors include esters, alkylamines, and amides.

Sol-gel processing is based on the hydrolysis and condensation of appropriate precursors, which, in most cases, involves the reaction of an alkoxide (either modified or unmodified) with water (i.e. the hydrolysis step). Water is thus used as the condensing agent. Ceramic materials that are suitable for use in the context of embodiments of the invention include, but are not limited to binary, ternary or quaternary ceramic materials, which can be carbides, nitrides, borides or oxides in various embodiments. Illustrative ceramic matrices include, for example, silicon carbide, tungsten carbide, chromium carbide (Cr₃C₂), titanium carbide (TiC), titanium nitride (TiN), titanium boride (TiB₂), aluminum oxide, silicon nitride (Si₃N₄), SiCN, Fe₂N, BaTiO₃, lithium aluminosilicate or mullite (a silicate mineral having two stoichiometric forms: 3Al₂O₃.2SiO₂ or 2Al₂O₃.SiO₂). silica, glass, glaze, copper oxide, lead oxide, aluminum oxide, titanium oxide, zirconium oxide, aluminum nitride, titanium nitride, zirconium nitride, silicon carbide and a mixture thereof.

The nanoparticles of the present invention may be also prepared by a dispersion polymerization synthesis. This polymerization method affords micron-size monodisperse particles in a single batch process. Dispersion polymerization may be defined as a type of precipitation polymerization in which one carries out the polymerization of a monomer in the presence of a suitable polymeric stabilizer soluble in the reaction medium. The solvent selected as the reaction medium is an appropriate solvent for both the monomer and the steric stabilizer polymers, but a non-solvent for the polymer being formed. Dispersion polymerization, therefore, involves a homogeneous solution of monomer(s) with initiator and dispersant, in which sterically stabilized polymer particles are formed by the precipitation of the resulting polymers. As a continuous medium, the properties of the solvent also change with increasing monomer conversion. Under favorable circumstances, the polymerization can yield, in a batch step, polymer particles of 0.1-15 mm in diameter, often of excellent monodispersity. In some embodiment, the nanoparticles of the present invention are synthesized using polymerization dispersion polymerization synthesis wherein, the stabilizer is a polyvinylpyrrolidone (PVP) and initiator is benzoyl peroxide (BP).

The following advantages are attributed to the nanoparticle compositions of the invention:
(i) Superior antibacterial effect compared to free triclosan;
(ii) Protection from any destroying chemical/biochemical environment during cell delivery, for example, UV radiation;
(iii) Water solubility, compared to the poor solubility of free triclosan.
(iv) Combined chemically stable/enzymatically (esterases) unstable urethane bond between both triclosan and the inorganic or organic matrix
(v) Enable controlled release of the biocide upon interaction with the pathogen through enzymatic activity.
(vi) Small particle size, typically up to 200 nm in diameter.
(vii) Minor leaching of the biocide from the nanoparticles in water.

The aforementioned advantages among others result with improved vehicle of triclosan that can be utilized to disinfect surfaces or as treatment of diseases or disorders in subjects in need thereof.

These and further embodiments will be apparent from the detailed description and examples that follow.

### EXAMPLES

### Example 1

### Preparation and characterization of triclosan-(3-(triethoxysilyl)propyl)carbamate (TTESPC).

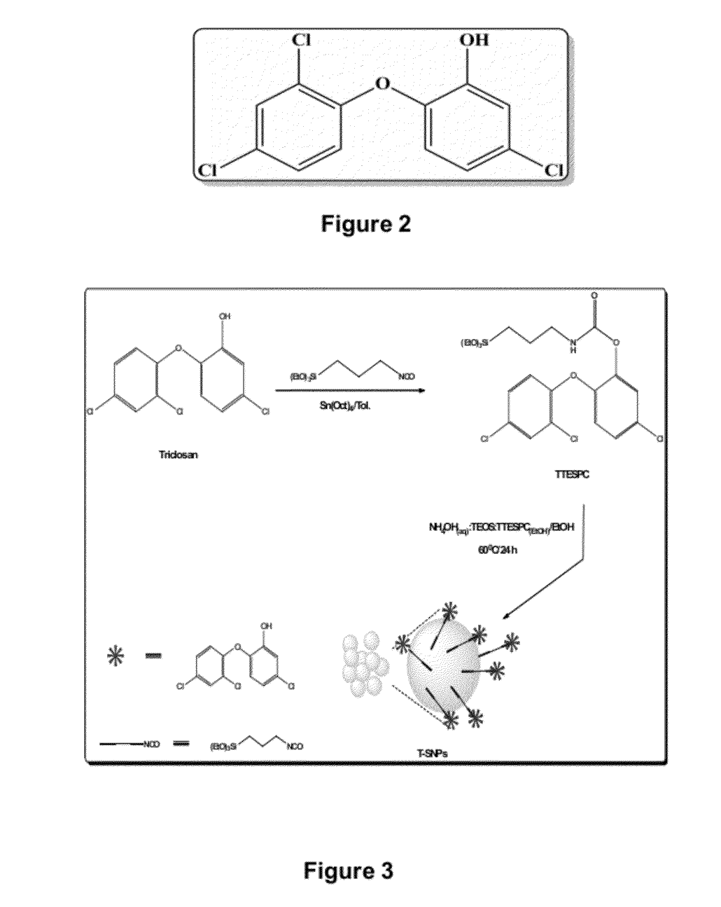

The synthesis of triclosan-(3-(triethoxysilyl)propyl)carbamate (TTESPC) was accomplished through a direct carbamoylation of triclosan with 3-isocyanatopropyltriethoxysilane in the presence of the Lewis acid, tetraoctyltin, as outlined in FIG. 3. Briefly, Triclosan (5-chloro-2-(2,4-dichlorophenoxy)phenol) (1 g, 3.45 mmol, 1 eq) and dry toluene (5.0 mL) were added to a three necked round-bottom flask under a N₂ atmosphere, so as to obtain a 0.7 M solution. 3-(Triethoxysilyl) propyl isocyanate (1.28 mL, 5.18 mmol, 1.5 eq) and tetraoctyltin (3.02 mL, 5.18 mmol, 1.5 eq) were added simultaneously to the reaction mixture, which was stirred at room temperature until no progress in the reaction could be observed by thinlayer chromatography (TLC) (4:1n-hexane:EtOAc) n-hexane:ethyl acetate (EtOAc). Toluene was evaporated until off-white oil emerged. Upon crystallization overnight, white crystals were obtained. These were filtered with cold n-hexane to remove traces of the stannane complex and dried under vacuum to yield 63.5% (1.17 g) of a white crystalline powder. All of the moisture-sensitive reactions were carried out in flame-dried reactions vessels. The melting points were determined using an electrothermal digital melting-point apparatus and were measured as mp 83.5±1° C.

The resulting TTESPC is a new compound designed specifically to introduce the triclosan moieties inside the inorganic silica matrix. The relatively facile and efficient synthesis allows for an easy scale-up process.

The proposed structure of the TTESPC was confirmed by 1H-NMR, 13C-NMR, IR, and UV-vis spectroscopy. ¹H-NMR and ¹³C-NMR spectra were obtained using a Bruker DPX 300 MHz spectrometer. The chemical shifts are expressed in ppm downfield from Me4Si (tetramethylsilane (TMS) used as an internal standard). The values are given using the δ scale. The presence of the aliphatic moieties upheld on the one hand (δ=3.81, 3.20, 1.63, and 1.21 ppm) and the aromatic peaks slightly shifted downfield and more clustered indicate a covalent attachment had indeed occurred. The broad triplet at 5.3 ppm due to the proton attached to the carbamate nitrogen further confirmed that the reaction took place. Multiplicities in the ¹³C-NMR spectra were determined by off resonance decoupling.

¹H NMR (300 MHz, CDCl₃, 25° C., TMS, δ) spectra of the resulting compound included bands in the following chemical shifts: 7.43 (d, J=2.5 Hz, 1H, ClCCHCl), 7.26 (d, J=2.5 Hz, 1H, ClCCHC(O)C(O)), 7.15 (dd, J=8.8, 2.5 Hz, 1H, ClCCHCHC(O)Cl), 7.13 (d, J=2.5 Hz, 1H, ClCCHCHC(O)C(O)), 6.87 (d, J=8.8 Hz, 1H, ClCCHCHC(O)C(O)), 6.83 (d, J=8.8 Hz, 1H, ClCCHCHC(O)Cl), 5.36 (bt, 1H, NH), 3.81 (q, J=7.0 Hz, 6H, CH₃CH₂O), 3.20 (m, 2H, NHCH₂), 1.63 (m, 2H, NHCH2C H₂), 1.21 (t, J=7.0 Hz, 9H, CH₃CH₂O), 0.62 (m, 2H, SiCH₂);

¹³C NMR spectra of the resulting compound included bands in the following chemical shifts (75.5 MHz, CDCl₃, 25° C., TMS, 6): 153.2 (1C, NHC═O(O)), 151.4 (1C, CHC(O)C(O)), 147.0 (1C, ClC(O)CH), 142.3 (1C, NHCO₂C), 130.2 (1C, ClCCHCl), 129.3 (1C, ClCCHC(O)), 129.1 (1C, ClCCHCHC(O)Cl), 128.1 (1C, ClCCHCHC(O)Cl), 126.4 (1C, ClCCHCHC(O)C(O)), 125.6 (1C, ClCC(O)CH), 124.8 (1C, ClCCHC(O)), 120.4 (1C, ClCCHCHC(O)Cl), 120.2 (1C, ClCCHCHC(O)C(O)), 58.5 (3C, CH₃CH₂O), 43.6 (1C, NHCH₂), 22.8 (1C, NHCH₂CH₂), 18.2 (3C, CH₃CH₂O), 7.5 (1C, SiCH₂);

IR (KBr) spectra of the resulting compound included as follows: ν=3320 (m; ν as (NH)), 2974 (m; ν as (CH₂)), 2927 (m; ν as (CH₂)), 2885 (m; ν as (CH₂)), 1717 (vs; ν (C═O)), 1534 (s), 1487 (s; ν as (aromatic C═C)), 1474 (vs), 1389 (w), 1280 (vs; ν as (SiOC)), 1250 (m), 1219 (m), 1187 (m), 1080 (vs; vs (phenolic CO)), 956 (m; ν as (aromatic CH)), 789 cm⁻¹ (m; ν as (CCl));

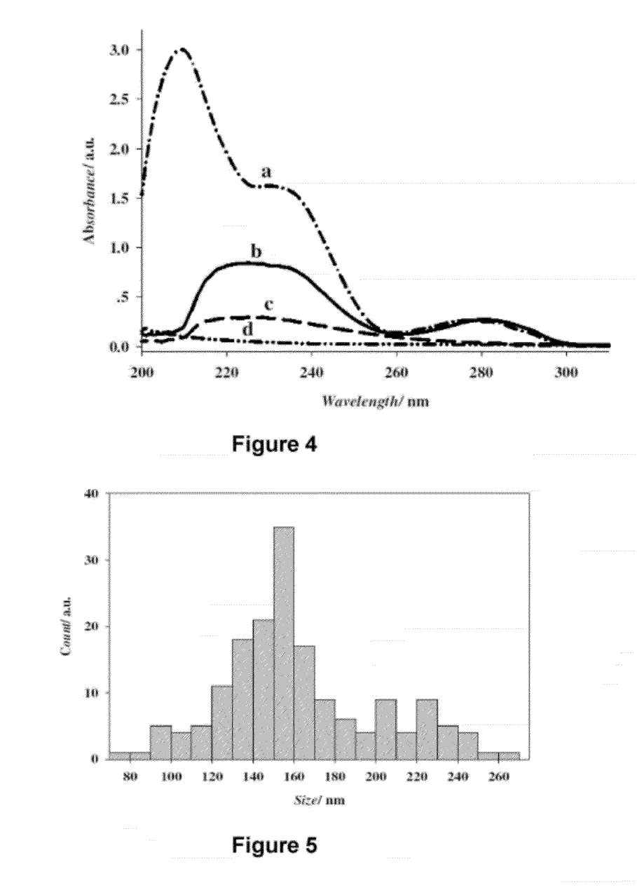

A UV-spectrum (CARY 100 Bio UV-vis spectrophotometer) UV-vis (Et₀H) included: λ max (e)=276 (2822), 230 (17 407), 209 nm (32 160) (FIG. 4).

Mass spectra, CIMS, of the resulting compound included the following parameters: (m/z (%)) 536.08 (M+5.48), 489.98 (C₂₀H₂₃Cl₃NO₅ Si., 100.00); HRMS (ESI, m/z): [M+H]+calculated for C₂₂H₂₈Cl₃NO₆ Si, 535.905; found, 536.085.

### Example 2

### Preparation and Characterization of Triclosan Silica Nanoparticles (T-SNPs)

Synthesis of T-SNPs was achieved through a series of experiments using a modified Stöber method (sol-gel synthesis; FIG. 3). 2.4 mL of a 28-30% solution of aqueous NH₄OH and 25 mL of HPLC grade absolute ethanol were added to a 100 mL vial containing a stirrer at a certain temperature (25° C. or 60° C., depending on the desired size of the nanoparticle). The medium was stirred for 5 min to obtain a homogeneous clear solution. Meanwhile, TTESPC (72.1 mg, 2.5% w/v) was dissolved completely in an additional 5 mL of ethanol and added to the previously described solution. The mixture was stirred for an additional 15 min in order to hydrolyze the silicate groups of the linker (TTESPC). Finally, 1.2 mL of TEOS was added to the clear solution, which was then stirred for 24 h at ambient temperature. T-SNPs of various sizes, size distributions and stabilities were prepared by changing the sol-gel process parameters (e.g., linker, base and TEOS concentrations), as well as the medium temperature. The resulting NPs were washed with EtOH using sequential centrifugation cycles (13,000 rpm) until a neutral pH was reached, then were washed twice with H₂O. Finally, the NPs were lyophilized to dryness.

Fourier Transform Infra Red (FTIR)-spectroscopy analysis was performed using a Brüker Equinox 55 FTIR spectrometer. The analysis was performed using 13 mm-diameter KBr pellets that contained 2 mg of the sample and 198 mg of KBr. The pellets were subjected to 200 scans at a resolution of 4 cm⁻¹. FIG. 1A shows the FTIR spectra of the linker (TTESPC) and FIG. 1B shows the FTIR spectra of T-SNPs and reveals the characteristic peaks of the functional groups present. The IR spectrum of TTESPC (FIG. 1) reveals absorption peaks at 3320 cm⁻¹, which corresponds to the carbamate NH asymmetric-stretching band, 2885-2974 cm⁻¹ (alkane CH₂ asymmetric stretching bands), 1717 cm⁻¹ (carbamate C═O stretching band), 1487 cm¹ (aromatic C═C stretching bands), 1280 cm⁻¹ (Si—O—C stretching bands), 1080 cm⁻¹ (phenolic symmetrical C-0 stretching bands), and 789 cm¹ (C—Cl stretching bands). The IR spectrum of the T-SNPs (FIG. 1B) shows a broad curve with a peak at 3390 cm¹, which corresponds to the carbamate NH and alkane CH₂ asymmetric-stretching bands, a peak at 1639 cm¹ (a red-shifted carbamate C═O stretching band), a broad curve with a peak at 1108 cm¹ (Si—O—C ether stretching bands, aromatic C═C stretching bands and phenolic symmetrical C—O stretching bands), a peak at 949 cm⁻¹ (aromatic C—H stretching bands), and a peak at 789 cm⁻¹ (C—Cl stretching bands). A wavenumber shift of the CO stretching (ν C═O) band gives insight into the molecular interaction occurring in the system under study. In particular, the shift to lower wavenumbers (“red-shift”) may be attributed to the presence of hydrogen-bond interactions with the carbamate carbonyl inside the inorganic solid SiO₂ matrix. The opposite effect, namely the shift to higher wavenumbers, (“blue-shift”) of the ν CH band of alcohols in polar organic compounds and surfactants observed for aqueous solutions, has also been observed.

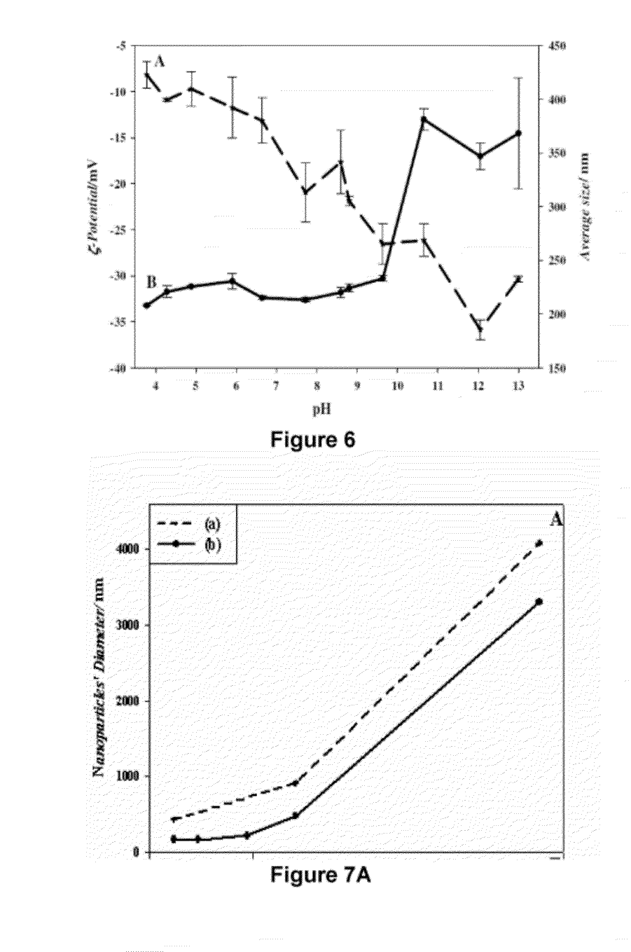

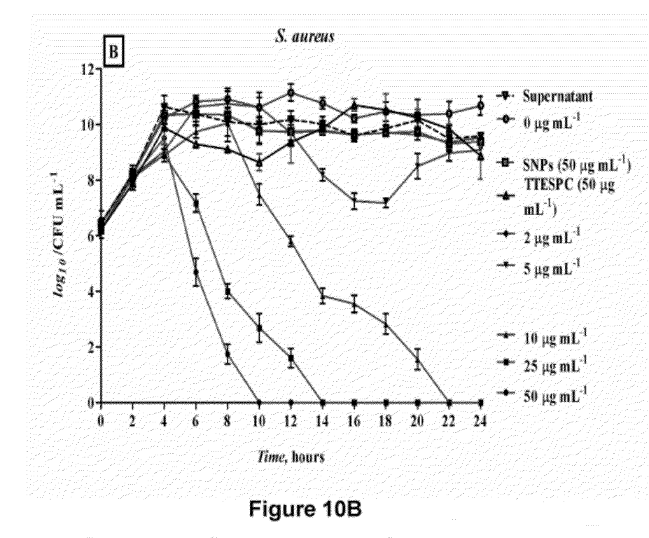

The hydrodynamic particle size, size distribution and ζ-potential of the particles were determined using a Zetasizer Nano series instrument (Nano-ZS, Malvern Instruments Ltd., UK) equipped with an MPT-2 multi-purpose titrator (Malvern Instruments Ltd., UK). ζ-Potential titration analyses were used in order to follow-up the colloidal stability of the hybrid-silica nanoparticles towards aggregation. DLS studies showed a hydrodynamic diameter of 164.3 nm (FIG. 5) which is in accordance with the actual TEM size (FIG. 9B) of similar dried particles, when considering the likely adsorption of water molecules onto the nanoparticle surface.

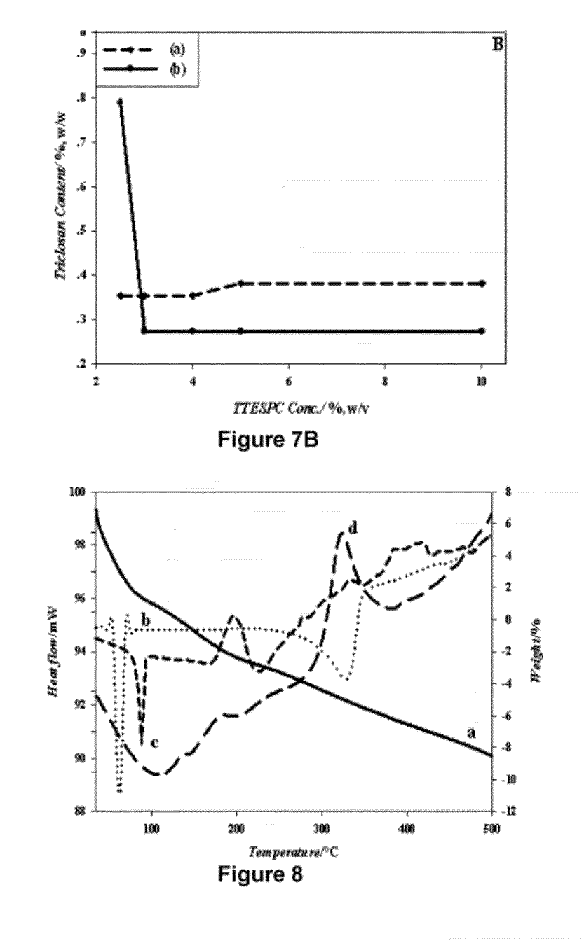

ζ-potential and particle-size titration versus rising pH values was performed in order to estimate the relative stability of the nanoparticles in aqueous media (FIG. 6). As can be observed, the ζ-potential increases in absolute value, from −8 mV at a pH of 3.7 to −36 mV at a pH of 12 (dashed line), which means that the particles became more stable. The ζ-potential is ca. −20 mV when the pH reaches a physiological value (pH=7.4). Interestingly, the linker (TTESPC) molecules, which are hydrophobic in nature, probably prefer to be oriented towards the inner part of the particle. Thus, the linker (TTESPC) molecules have minimal influence on the net surface potential of the nanoparticles. Further evidence of the stability of these particles is the lack of aggregation over a relatively wide pH-value range, since the measured average diameters of the T-SNPs (solid line) remain practically stable until a pH of 10, disclosing 150-170 nm values (i.e., very close to their TEM-measured diameter) (FIG. 9B).

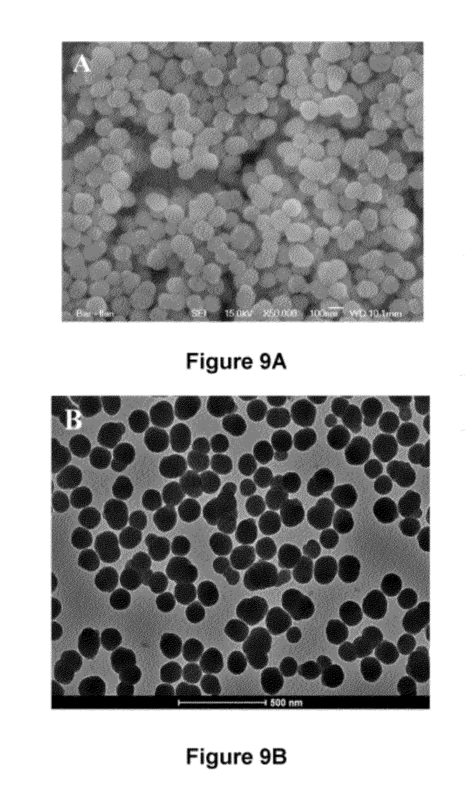

Nanoparticle size measurements showed a correlation between the average nanoparticle diameter (measured by DLS) and the TTESPC's initial concentration at 25° C. (dashed line) and at 60° C. (solid line) (FIG. 7A). By reducing the linker's (TTESPC) initial concentration from 10 to 2.5 wt %, the particle's diameter started stabilizing at a 200 nm value until reaching a final size of 160 nm. The optimal compromise between the final triclosan weight percent inside the nanoparticles and their corresponding diameter was obtained when an initial concentration of 2.5% (w/v) of the linker (TTESPC) was added to the reaction mixture. In order to estimate the amount of linker present in the nanoparticles, a measurable molecular entity could be used as an internal standard. For this purpose, measurable quantities of chlorine (Cl) could be directly correlated to the amount of incorporated triclosan: its quantity was determined by elemental analysis. FIG. 7B describes the correlation between the initial linker concentration and the triclosan weight percentage inside the T-SNPs as a function of synthesis temperature. The dashed curve is for syntheses performed at 25° C., for which no dependency of the triclosan content on the TTESPC concentration was observed. The solid curve relates to syntheses performed at 60° C. One can observe that as the linker percentage diminishes and reaches a certain value, (2.5% w/w), the triclosan content inside the T-SNPs rises to 0.79 wt %. The amount of triclosan was calculated from the Cl quantity that was measured by elemental (chlorine) analysis that was performed using a combination of an oxygen-flask combustion technique and subsequent ion chromatography (DIONEX). The relative quantity of triclosan was calculated according to Equation 1: mol_{f} (Cl)/3×289.64 g/mol (Mw of triclosan)=% triclosan inside the T-SNPs. In Equation 1, mol_{f} indicates the amount of chlorine in moles found by elemental analysis. The expression is divided by 3 due to the presence of 3 chlorine atoms in the triclosan molecule. If one assumes that the amount of chlorine found relates to an arbitrary 100 mass units, the expression result indicates percentages. The rather low final concentration of triclosan in the nanoparticles was probably due to the relatively high hydrophobicity of the TTESPC, which hinders the incorporation of this sterically demanding linker into the midst of the inorganic, hydrophilic silica matrix.

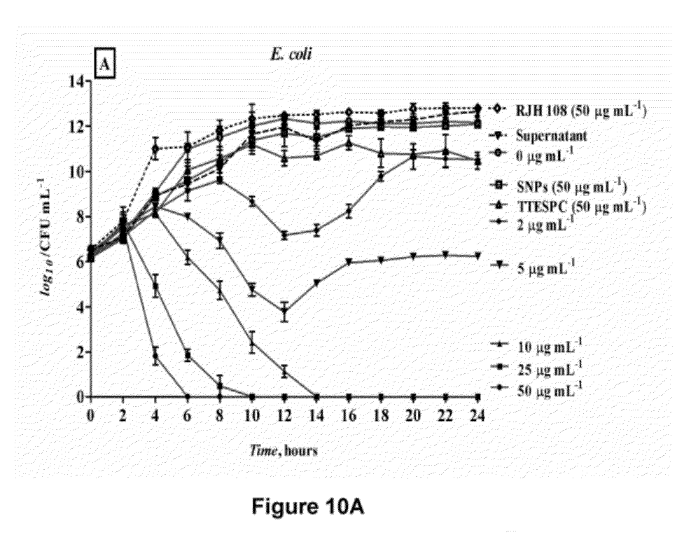

Thermal measurements were performed by thermogravimetric-analysis (TGA) and differential scanning calorimetry (DSC). The TGA measurements were carried out using a TA Instruments apparatus (1GA Q500 model) for TGA, and DSC using a METTLER TOLEDO DSC 822e instrument, with a 25-500° C. temperature profile (10° C. min⁻¹, N₂ atmosphere, 100 mL min⁻¹) for both. TGA afforded the temperature profiles of the hybrid-silica particles versus TTESPC. DSC revealed the endothermic and exothermic processes involving the hybrid-silica particles versus the linker during the heating process. FIG. 8 illustrates the thermal analyses performed on the T-SNPs. Curve a corresponds to the TGA thermogram (using a 25-500° C. temperature profile; 10° C. min⁻¹, N₂ atmosphere, 100 mL min⁻¹). This thermogram consists of several slopes, with one main-step slope showing approximately 3% weight loss (at approximately 100° C.), which may correspond to a loss of water molecules entrapped in the inorganic matrix. The second slope, between approximately 100 and 190° C. (approximately 2% weight loss) may correspond to a loss of the water molecules that participate in the hydrogen bonding between the silanol groups on the surface and near the surface and a release of CO gas from the nanoparticles as their degradation starts. This fact may be corroborated with the DSC thermogram (see the long-dashed curve d, FIG. 8), with two proximal exothermic peaks between 110 and 200° C. Afterwards, the degradation of the organic content begins, as evidenced by the moderate weight loss in the TGA curve (approximately 6.5% weight loss) and the moderate exotherm in the DSC thermogram. The exotherm with its peak at 320° C. may be attributed to the formation of radicals created as a result of chlorine-radical combination into Cl₂. The same peak appears in the DSC thermogram of the linker itself but shifted to a much lower temperature (approximately 190° C.). This can be explained by the fact that the inorganic matrix shields and protects the entrapped linker molecules from the external environment, resulting in a higher degradation temperature. Curves b (dotted curve) and c (short-dashed curve) correspond to DSC thermograms of triclosan and of the linker, TTESPC, respectively. One can clearly see one sharp endotherm in each curve, with peaks corresponding to the melting points of triclosan and TTESPC (i.e., 56° C. and 83° C. in curves b and c respectively). There is another endotherm, at approximately 344° C., corresponding to the boiling point of triclosan. Beyond this point, the nanoparticle decomposition begins. The lack of the peaks mentioned before in curve d further emphasizes the covalent attachment of the linker to the inorganic matrix, as well as the absence of the free biocide or linker within the inorganic matrix of the T-SNPs.

High-resolution-SEM and energy-dispersive-spectroscopy (EDS) analyses were carried out on a JEOL JSM-7000F instrument. TEM and HR-SEM analyses enabled the determination of the morphology, size and size distribution of the particles, while EDS and elemental analyses provided particle-composition data. The TEM micrographs were taken using a Tecnai Spirit instrument (120 kV). Samples for TEM were prepared by placing a drop of the diluted spheres dispersed in an (50% v/v) ethanol-water solution on 400 mesh carbon-covered Cu grids Pk/100 (SPI Supplies West Chester, USA) and then air-drying them. The average diameter, particle-size distribution, and surface morphology of the particles were obtained by SEM or TEM, followed by a statistical analysis (ImageJ software) by measuring at least 200 particles for each sample. The SEM samples were coated with a thin layer of gold by a sputtering deposition technique. FIG. 9A shows scanning-electron-microscopy (SEM) and FIG. 9B shows transmission-electron-microscopy (TEM) micrographs of the T-SNPs obtained in a typical experiment at 60° C. with 2.5% (w/v) of TTESPC. It can be appreciated from these micrographs the smooth, spherical morphology of the nanoparticles. These nanoparticles were obtained with a narrow size distribution and an average diameter of 130±30 nm.

### Example 3

### Preparation of TA-NPs

Triclosan acrylate nanoparticles (TA-NPs) were obtained from 5-chloro-2-(2,4-dichlorophenoxy)phenyl acrylate (TA) as detailed herein. To a refluxing solution of triclosan[5-chloro-2-(2,4-dichlorophenoxy)phenol] (1 eq, 6 gr, 20.7 mmol) in dry toluene (20 mL) were added dropwise in the course of 30 minutes triethylamine (2.8 eq, 7.9 mL, 56.7 mmol) and acryloyl chloride (1.2 eq, 2 mL, 24.7 mmol). The end of the dropwise addition was characterized by a change of the colorless solution to an orange-brownish suspension. The mixture was stirred overnight at reflux conditions. The solvent was evaporated to obtain an orange-brownish tar, which was directly subjected to a column chromatography (9:1 ether:EtOAc). The product, TA, was obtained as clear oil in 83% yield (5.88 g).

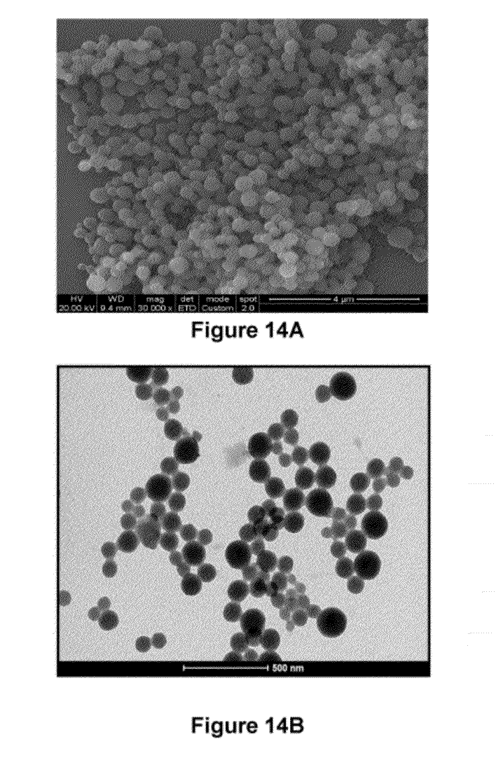

Poly(Triclosan acrylate) (PTA) was prepared by a typical procedure of a dispersion polymerization. Nanometer-sized PTA particles with an average diameter of 95±20 nm were formed by dissolving 0.4 g of TA, 10 mg BP, and 0.1 g PVP in 7.2 mL of ethanol and 3.0 mL of 2-methoxyethanol. The total volume of the solution was 10.2 ml and the TA, BP and PVP concentrations were 4, 0.1 and 1% (w/v), respectively. For the polymerization of TA, the vial was shaken at 73° C. for 6 h. The resulting particles were washed by 6 intensive centrifugation cycles (13,000 rpm) with ethanol and then dried under vacuum at 400° C. PTA nanoparticles of various sizes, size distributions and stabilities were prepared by changing the polymerization parameters, e.g., TA and initiator concentrations, type and concentration of the polymeric surfactant, time of reaction of the polymerization system, and co-solvent concentration. FIG. 13 is a scheme demonstrating the structure of the polymer TA-NPs.

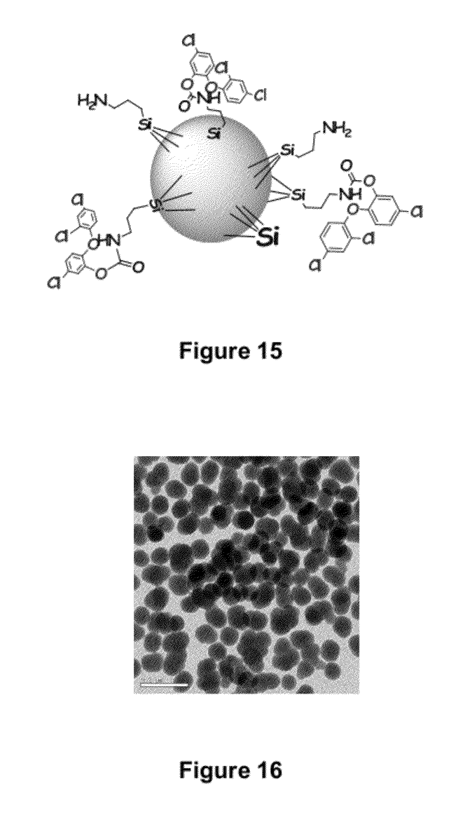

The average diameter, TA particle-size distribution, and surface morphology of the particles were obtained by SEM (FIG. 14A) or TEM (FIG. 14B). It can be appreciated from these micrographs the smooth, spherical morphology of the nanoparticles. These NPs were obtained with a narrow size distribution and an average diameter of 150±30 nm.

### Example 4

### Antimicrobial Activity

In order to investigate the antimicrobial activity of the nanoparticles, a series of biological experiments were designed and carried out on two common bacterial pathogens, Escherichia Coli (E. coli) (Gram-negative) and Staphylococcus Aureus (S. aureus) (Gram-positive). Killing curves were determined in triplicate using starting inocula of 10⁶ CFU mL⁻¹. Fresh, overnight growths of bacteria in TSB or TSB-Glu were diluted as necessary to produce the desired starting inocula in 10 mL of medium. T-SNPs and triclosan were tested in concentration ranges from 0 to 50 μg mL⁻¹ and 0 to 1 μg mL⁻¹, respectively. Unloaded SNPs were added at the highest tested concentration (50 μg mL⁻¹). TTESPC was previously dissolved in dimethyl sulfoxide (DMSO) and added to the media at a concentration of 50 μg mL⁻¹. Samples (100 μL) were removed from each well every 2 h and diluted appropriately in saline. Colony-forming units were determined by spotting 5 μL samples in triplicate on Luria-Bertani agar plates after 24 h incubation at 37° C. To confirm that the antimicrobial properties of the T-SNPs were mediated by the sole release of triclosan, the triclosan-resistant strain of E. coli, RJH108, was exposed to the highest concentration of T-SNPs tested (50 μg mL⁻¹). The viability was determined by using the same experimental protocol as described above.

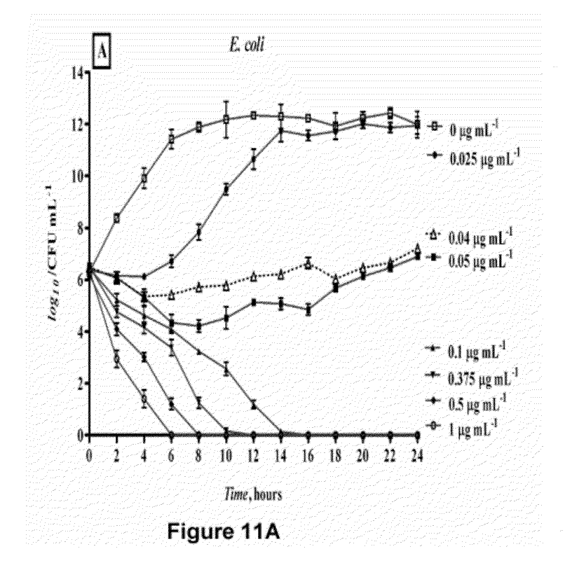

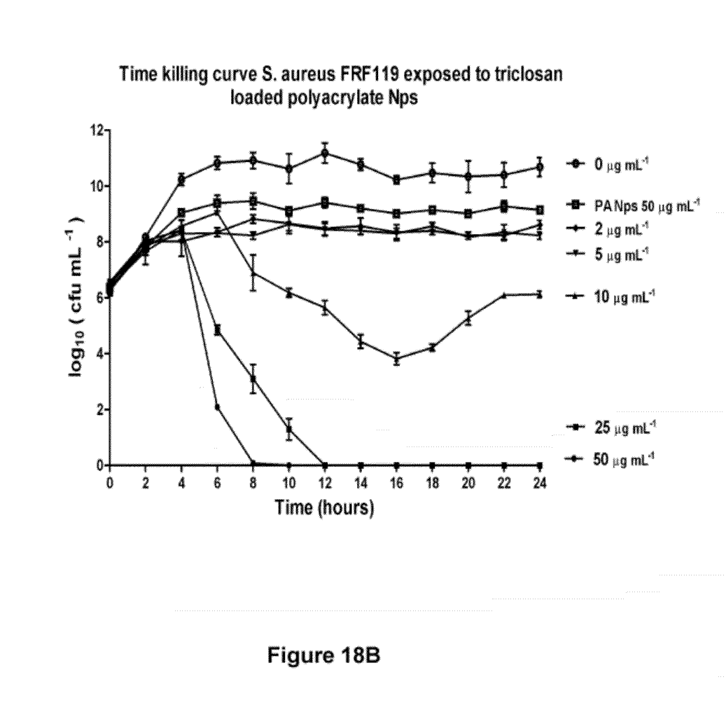

FIGS. 10 and 18 depict the antimicrobial activity of the T-SNPs and the TA-NPs against two common E. coli (FIG. 10A and FIG. 18A, respectively) and S. Aureus (FIG. 10B and FIG. 18B, respectively).

FIG. 10 demonstrates E. coli and S. aureus grown at the following concentrations of T-SNPs: 2 μg mL⁻¹ (closed diamonds), 5 μg mL⁻¹ (inverted closed triangles), 10 μg mL⁻¹ (small closed triangles), 25 μg mU⁻¹ (closed squares) and 50 μg mL⁻¹ (closed circles). Untreated bacteria (opened circles), 50 μg mL⁻¹ of unloaded nanoparticles (opened squares) and TTESPC at 50 μg mL⁻¹ (large closed triangles) served as controls, in which no antibacterial activity was observed. The results presented demonstrate that, for the two types of bacteria, the T-SNPs caused a reduction in growth in a dose-dependent manner and the minimal bactericidal concentration (MIC) measured was 10 μg mL⁻¹ for both E. coli and S. aureus. Despite the similar MICs, a difference in the sensitivity of these strains to the T-SNPs treatment was observed. E. coli seems to be more sensitive compared with S. aureus, and complete killing was observed after 14 h, whereas complete killing of S. aureus was observed only after 22 h. Next, in order to rule out the possibility of the leaching of triclosan from the nanoparticles (even without the presence of bacteria), which would suggest that the T-SNPs are unstable, the effect of growth media preincubated with the nanoparticles was evaluated. A high concentration of T-SNPs (50 μg mL⁻¹) was incubated in a growth medium (without bacteria) for 24 h (same conditions as for the killing experiments mentioned below). Then, the nanoparticles were centrifuged, filtered and the supernatant was incubated separately with E. coli and S. aureus and their growth was monitored. It can clearly be observed from the plotted graphs (FIGS. 10A, and 10B, dashed curves with inverted opened triangles) that the growth rates of the two pathogens were not impeded and resembled the control. This observation strongly suggests that the activity required the presence of bacteria and that either no triclosan was released from the T-SNPs in the first incubation step or the amount of released triclosan remained very low and was not sufficient to impede bacterial growth.

To further validate that the antimicrobial properties of the T-SNPs were solely mediated by the release of triclosan, a second series of experiments was done. In this experiment, a triclosan-resistant E. coli RJH108 strain was grown with the highest tested concentration of T-SNPs (50 μg mL⁻¹). As can be seen, RJH108 strain was not affected by the presence of the T-SNPs (dotted line with opened diamonds, FIG. 10A).

Similar antimicrobial results were obtained using the TA-NPs on E. Coli (FIG. 18A) and on S. aureus (FIG. 18B). E. coli and S. aureus were grown at various concentrations of TA-NPs, as follows: 2 μg mL⁻¹ (closed diamonds), 5 μg mL⁻¹ (inverted triangles), 10 ng mL⁻¹ (triangles), 25 μg mL⁻¹ (closed squares) and 50 μg mL⁻¹ (closed circles). Untreated bacteria (opened circles) and 50 μg mL⁻¹ of unloaded nanoparticles (opened squares) served as controls, in which no antibacterial activity was observed.

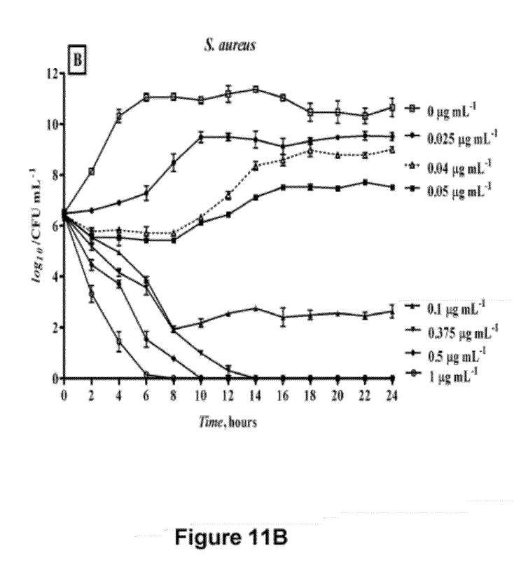

The MIC of free triclosan that affected the growth of the two types of bacteria was measured (FIGS. 11A-B). E. coli (FIG. 11A) and S. aureus (FIG. 11B) were grown at various concentrations of free triclosan: 0.025 μg mL⁻¹ (closed circles), 0.04 μg mL⁻¹ (opened triangles), 0.05 μg mL⁻¹ (closed squares), 0.1 μg mL⁻¹ (closed triangles), 0.375 μg mL⁻¹ (inverted triangles), 0.5 μg mL⁻¹ (diamonds) and 1 μg mL⁻¹ (opened circles). Untreated bacteria (opened squares) served as control, in which no antibacterial activity was observed. MIC of free triclosan was demonstrated at 0.1 μg mL⁻¹. A similar killing-curve pattern was observed with the T-SNPs, yet with a significant distinction. As calculated from the elemental analysis, the triclosan constituted only 0.79 wt % of the nanoparticles, meaning that the highest concentration of nanoparticles taken for the experiments, 50 μg mL⁻¹, encompassed only 0.041 μg mL¹ of covalently bound triclosan. At this concentration, the free triclosan only began to impede the bacterial growth, whereas the T-SNPs killed all of the bacteria. At this concentration of triclosan (≈0.04 μg mL⁻¹, dotted lines in FIG. 11A-B) the T-SNPs afforded an increase of 5-6 log in killing capacity compared with the free biocide. It is likely that the strong antimicrobial effect stems from the protection of the harsh chemical and/or biochemical environment provided by the nanoparticles during delivery. For example, silica matrices are known to provide UV protection (Zhang Y. et al., 2010, Colloid Surf, 353, 216). This means that these matrices prevent the UV-sensitive triclosan from UV-induced decomposition to deleterious dioxins. Furthermore, the T-SNPs may deliver a local high concentration of triclosan near the bacteria via membrane attachment, thus increasing its overall efficacy.

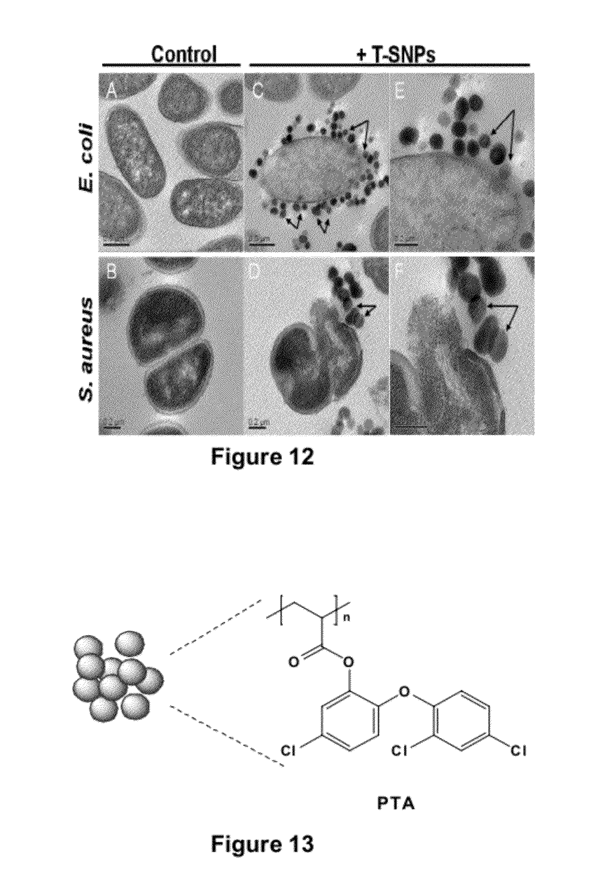

To examine the possibility that the T-SNPs intimately interact with the bacteria, TEM measurements were conducted to investigate the mode of action of the nanoparticles on the tested cells (FIG. 12). Samples of the E. coli and S. aureus cultures were centrifuged and washed immediately after 2 or 4 h (for E. coli and S. aureus, respectively) of treatment with and without T-SNPs (50 μg mL⁻¹). The samples were then fixed in 25% pentane-1,5-dial/polyoxymethylene in a cacodilate buffer at room temperature for 1 h. Then, the samples were washed with the same cacodilate buffer and fixed in 1% osmium tetraoxide (OsO₄). Sample embedding was carried out using a standard protocol (polymerized beads of agar resin) and 60 nm-thick slices were cut with a diamond knife (LBR ultratome III). The resulting slices were deposited on bare 200 mesh copper grids, and stained with 2 wt % uranyl acetate for 5 min. Finally, the grids were dried in a desiccator and examined using a Fei Tecnai g2 instrument at 120 kV.

Untreated E. coli (FIG. 12A) and S. aureus (FIG. 12B) cells both showed the normal cell morphology, possessing the distinct cell walls and membrane structures typical of Gram-negative and Gram-positive bacteria. Quite interestingly, the T-SNP-treated samples of both E. coli (FIG. 12C) and S. aureus (FIG. 12D) showed that the interacting T-SNPs were localized either on the cell surface or within the cell membrane, causing a pronounced damage to the cell walls of E. coli (FIG. 12E) and S. aureus (FIG. 12F). Although no nanoparticle internalization was detected, the imaging results reinforce the importance of direct nanoparticle-bacteria interactions for the promoted antibacterial activity. In this manner, TEM analyses further suggests a possible mechanism of action of the T-SNPs, emphasizing the importance of the role played by membrane-associated enzymes in the triclosan-release phase.

### Example 5

### Preparation and Characterization of Aminated Hybrid Silica Nanoparticles Containing Triclosan (NH₂-T-SNPs)

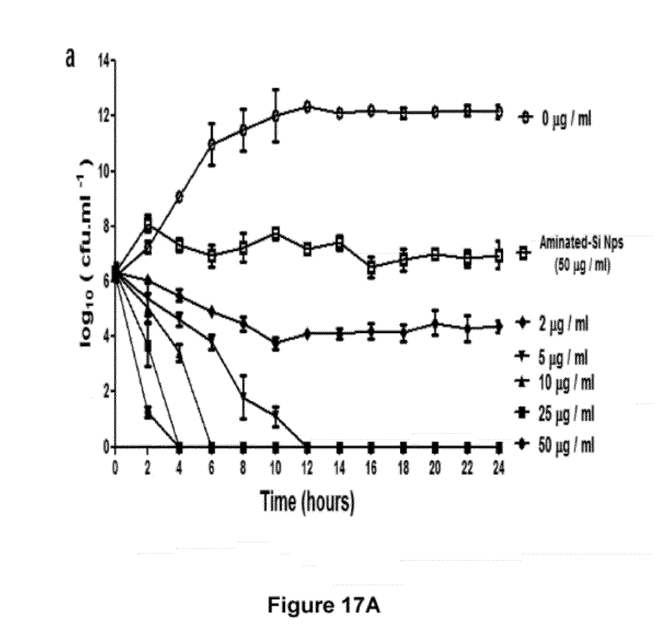

T-SNPs (triclosan-loaded silica nanoparticles) were re-dispersed in 150 mL EtOH using an ultrasonic bath. Then 1.5 mL of (3-aminopropyl)triethoxysilane (APTES) was added and the suspension stirred at room temperature overnight. The resulting nanoparticles (aggregates) were washed by intensive centrifugation cycles (12,500 rpm, RT) with EtOH and lyophilized to dryness. FIG. 15 is a scheme demonstrating the chemical structure of aminated hybrid silica nanoparticles containing triclosan (NH₂-T-SNPs).

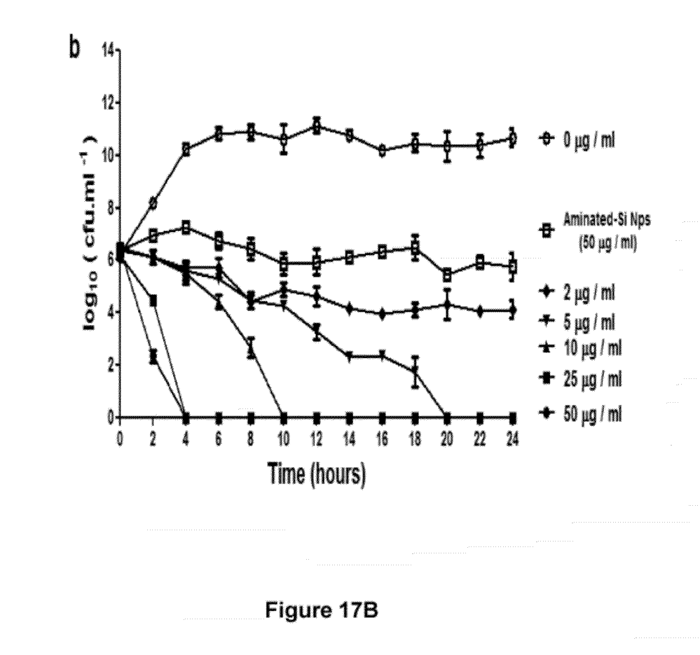

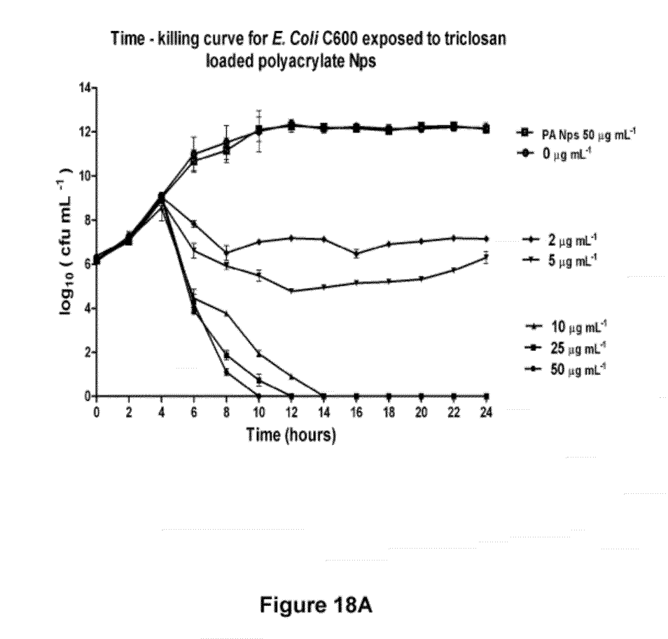

It was found that the average diameter of the single particles, as was calculated from TEM studies is 71.2±8 nm (FIG. 16). The ζ-potential after nanoparticle amination was +22.4 mV (positive value). To further validate the presence of the polyamine group shell on the surface of these newly formed nanoparticles, a Kaiser test (an amine quantification test) was performed. It was found that there is an average of 0.156 mmol g⁻¹ of free amine groups on the nanoparticle surface. The antimicrobial activity of NH₂-T-SNPs was studied in E. Coli (FIG. 17A) and on S. aureus (FIG. 17B). E. coli and S. aureus were grown at various concentrations of NH₂-T-SNPs, as follows: 2 μg mL⁻¹ (closed diamonds), 5 μg mL⁻¹(inverted triangles), 10 μg mL⁻¹ (triangles), 25 μg mL⁻¹ (closed squares) and 50 μg mL⁻¹ (closed circles). Untreated bacteria (opened circles) and 50 μmL⁻¹ of unloaded nanoparticles (opened squares) served as controls, in which no antibacterial activity was observed. The biological studies of NH₂-T-SNPs show that there is no significant difference in the overall activity of the aminated (FIG. 17A-B) as compared to non aminated nanoparticles (FIG. 10A-B), but the lag-time of their antibacterial activity is reduced to 0. As can be seen in FIG. 17, NH₂-T-SNPs nanoparticles have immediate antimicrobial activity following interaction with the pathogen cells.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A triclosan carbamate derivative.

2. The triclosan carbamate derivative of claim 1, comprising a silane moiety.

3. The triclosan carbamate derivative of claim 2, comprising triclosan and an isocyanate silane moiety.

4. The triclosan carbamate derivative of claim 2, consisting of 5 chloro-2-(2,4-dichlorophenoxy)phenyl 3-isocyanatopropyltriethoxysilane.

5. The triclosan carbamate derivative of claim 1 characterized by one or more of the following properties: a Fourier Transform Infra Red spectrum as set forth in FIG. 1, and a melting point of 83.5° C.±1° C.

6. A composition comprising a plurality of nanoparticles, each nanoparticle comprising a triclosan derivative and a carrier.

7. The composition of claim 6, wherein the plurality of nanoparticles exhibit an average particle size diameter within the range of 30 nm to 200 nm.

8. The composition of claim 6, wherein the triclosan derivative comprises a triclosan carbamate silane moiety.

9. The composition of claim 8, wherein the triclosan carbamate derivative comprises 3-isocyanatopropyltriethoxysilane.

10. The composition of claim 6, wherein the carrier is an inorganic carrier.

11. The composition of claim 10, wherein the inorganic carrier is a ceramic matrix.

12. The composition of claim 10, wherein the inorganic carrier is a solid silica matrix.

13. The composition of claim 10, wherein the triclosan carbamate derivative is covalently bound to the inorganic carrier.

14. The composition of claim 10, wherein the inorganic carrier is positively charged.

15. The composition of claim 13, further comprising a positively charged moiety selected from the group consisting of: polyethyleneimine, polyglutamic species, positively charged polysaccharides, chitosan and silicate derivatives thereof.

16. The composition of claim 14, wherein the inorganic carrier comprises polyaminated silica shells.

17. The composition of claim 6, wherein the amount of triclosan is within the range of 0.25 to 1 wt. % relative to the weight of the nanoparticle.

18. The composition of claim 6, wherein each nanoparticle comprises triclosan acrylate polymer.

19. The composition of claim 6, for inhibiting microbial growth.

20. A method of inhibiting microbial growth comprising contacting a surface with the composition of claim 6, thereby disinfecting said surface.
